(19) United States
(12) Patent Application Publication
(10) Pub. No.: US 2013/0214024 A1
(43) Pub. Date: Aug. 22, 2013

(54) Introducer System and Assembly for Surgical Staplers

(71) Applicant: Hidehisa Thomas Takei, Riverside, CA (US)

(72) Inventor: Hidehisa Thomas Takei, Riverside, CA (US)

(21) Appl. No.: 13/739,057

(22) Filed: Jan. 11, 2013

Related U.S. Application Data
(63) Continuation-in-part of application No. 12/756,713, filed on Apr. 8, 2010.

(51) Int. Cl. A61B 17/068

(52) U.S. Cl. CPC A61B 17/068
USPC 227/175.1

(57) ABSTRACT

An introducer assembly for use with a surgical stapler is provided. The introducer assembly includes an elongated main body portion, one or more mounting mechanisms associated with the main body portion, and a plurality of petals or fingers positioned at a distal end of the elongated main body portion. The one or more mounting mechanisms are adapted to detachably mount with respect to the surgical stapler. The plurality of petals or fingers define a distal junction and a proximally-directed mounting member that is adapted to detachably engage a distal portion of the surgical stapler. Exemplary introducer assemblies may include an adjustable length of the plurality of petals or fingers to conform to a distal end of the surgical stapler, an adjustable height of the proximally-directed mounting member to adjust an entry angle, means for detachably engaging a trocar, and/or means for insufflating air into a passage.

### CROSS-REFERENCE TO RELATED APPLICATIONS

This application claims is a continuation-in-part application that claims the benefit of co-pending non-provisional patent application entitled “Introducer System and Assembly For Surgical Staplers” which was filed on Apr. 8, 2010, and assigned Ser. No. 12/756,713. The entire content of the foregoing application is incorporated herein by reference.

### BACKGROUND

1. Technical Field

The present disclosure is directed to a system/assembly for facilitating introduction and/or positioning of a surgical stapler at or to a desired anatomical location. More particularly, the present disclosure is directed to an introducer system/assembly that is adapted to be detachably mounted with respect to a surgical stapler, e.g., an end-to-end or end-to-side anastomotic stapler.

2. Background Art

Wound closure techniques include the use of surgical staplers and clip appliers in many clinical applications. Surgical staplers and clip appliers take many forms and are adapted for use in various modalities. Thus, for example, certain staplers are specifically designed for application of skin staples, while others are adapted for use in internal procedures. Indeed, surgical staplers and clip appliers have been developed and are employed in both “open” and “minimally invasive” surgical procedures.

One particular form of surgical stapler having a specialized design and specialized uses is a circular stapler for use in performing end-to-end and end-to-side anastomotic procedures, e.g., the EEA™ line of staplers (Covidien, Inc., Mansfield, Mass.). The noted circular staplers generally take the form of a tubular instrument that defines a tubular shaft at a distal end thereof. A disposable cartridge is generally adapted to be mounted with respect to tubular shaft, such cartridge containing two or more circular rows of staggered staples, pushers for driving the staples from the cartridge and a circular knife within the inner ring of staples. An anvil assembly is adapted to mount with respect to a distally extending rod such that, when the anvil assembly is brought into close juxtaposition with the staple cartridge, the staples may be discharged from the cartridge and formed against anvil pockets defined in the anvil assembly. In addition, the anvil assembly also generally includes a plastic ring into which the circular knife is driven during the stapling operation. A control mechanism is provided at the proximal end of the surgical stapler to control relative movement between the staple cartridge and the anvil assembly. Once approximated, an actuating mechanism at the proximal end of the surgical stapler is employed to deliver staples into tissue and advance the circular knife into the plastic ring, thereby forming an anastomotic junction.

Circular staplers of the type described above are generally employed in esophageal and rectal procedures, although the staplers have utility throughout the gastrointestinal tract. For example, in bowel surgeries, purse-stringed bowel segments to be anastomosed are passed over the anvil assembly—which is typically dome-shaped—and the staple cartridge, respectively, and the purse-strings tightened around the central shaft. The anvil assembly and staple cartridge are then approximated and upon actuation of the actuating mechanism, an instantaneous, minimally-inverting, end-to-end or end-to-side anastomosis is effectuated. The circular stapler may be introduced and positioned through a natural orifice, e.g., the anus (e.g., a low anterior resection of the rectum) or the mouth (e.g., high esophago-gastric anastomosis). Alternatively, the circular stapler can be introduced through an opening formed during a procedure, e.g., in the stomach for esophagogastrostomy, in the small bowel for esophagojejunostomy, the terminal ileum for esophagocecostomy, and the like.

Of note, introduction and positioning of a circular stapler can be difficult in practice due to the physical structures associated with the distal end of the stapler. In particular, the stapler cartridge generally defines a substantially flat surface that is to be advanced through the intestinal tract or other anatomical lumen. Indeed, challenges exist in introducing a substantially flat surface into an orifice and thereafter advancing such flat surface through the lumen, e.g., up into the rectum, potentially encountering redundancies to the colon, redundant mucosa and/or prominent valves. These challenges can be further complicated by the varying configurations and/or sizes of surgical staplers, e.g., the surgical stapler head, and the like. The folds and/or texture of the inner surface of the lumen may create further challenges to advancing the surgical stapler. Difficulties in introducing a surgical stapler and/or positioning the surgical stapler in a desired anatomical location can inhibit its successful use.

Prior art efforts to address the above-noted issues have been less than satisfactory. Thus, for example, U.S. Pat. No. 5,404,870 to Brinkerhoff et al., U.S. Pat. No. 5,836,503 to Ehrenfels et al., and U.S. Patent Publication No. 2005/0165438 to Gritsus disclose devices and/or geometries that are intended to facilitate introduction and/or positioning of surgical staplers. Additional patent-related publications of background interest include U.S. Pat. Nos. 7,318,830; 5,355,897; 5,314,436; 4,471,782; 3,672,367; and 2,007,626, as well as U.S. Patent Publication No. 2005/0236459.

However, despite efforts to date, a need remains for introducer systems and assemblies that facilitate introduction and positioning of a surgical stapler relative to a desired anatomical location. A need further remains for introducer systems and assemblies that are adapted for easy and reliable detachment from the surgical stapler once its introduction and/or positioning functionalities are complete. These and other needs are satisfied by the introducer systems and assemblies disclosed herein.

### SUMMARY

According to the present disclosure, an advantageous device, assembly and method are provided for facilitating introduction and/or positioning of a surgical stapler relative to a desired anatomical location. The disclosed device/assembly takes the form of a “tear-away” introducer that is adapted to be detachably mounted with respect to the surgical stapler. In exemplary embodiments, the disclosed introducer includes a distal component that defines a plurality “petals” or “fingers” that (i) come together in a mounting member at their distal end, and (ii) cooperate with an elongated main body portion at their proximal end. The petals/fingers define an inner region that is configured and dimensioned to accommodate the distal end of a surgical stapler, thereby facilitating anatomical introduction of the circular stapler. The elongated main body portion of the disclosed introducer defines mounting mechanism(s), e.g., “C-shaped” mounting brackets, that are adapted to detachably engage the outer surface/shaft of the surgical stapler.

In an exemplary embodiment, the disclosed introducer is generally mounted with respect to a surgical stapler with the distal mounting member positioned relative to a distal component of the surgical stapler. The mounting mechanisms, e.g., C-shaped brackets of the introducer, detachably engage the stapler shaft. In advancing the surgical stapler to the desired stapling site, the petals/fingers of the introducer help to guide the stapler past potential obstructions and/or anatomical irregularities. Thereafter, the introducer is adapted to be detached from the stapler by pulling proximally on the elongated main body portion, causing the petals/fingers to “tear away” from around the distal portion of the surgical stapler, thereby facilitating removal from the stapler.

In an alternative embodiment, the disclosed introducer may include or cooperate with one or more intermediate structures, e.g., extension member(s). For example, one or more intermediate structures may be positioned between the disclosed petals/fingers and the elongated main body portion. Of note, the intermediate structure(s) may include structural features and/or functionalities that facilitate interaction with a surgical stapler, e.g., mounting mechanism(s) for detachably securing the disclosed introducer with respect to a surgical stapler.

In accordance with embodiments of the present disclosure, exemplary introducer assemblies for use with a surgical stapler are provided that generally include an elongated main body portion. The exemplary assemblies generally include one or more mounting mechanisms associated with the elongated main body portion. The one or more mounting mechanisms can be configured and dimensioned for detachably mounting with respect to the surgical stapler. The exemplary assemblies generally include a plurality of petals or fingers defining a distal junction and a proximally-directed mounting member. The proximally-directed mounting member can generally be configured and dimensioned to detachably engage a distal portion of the surgical stapler. A length of the plurality of petals or fingers can be adjustable to conform to a distal end of the surgical stapler.

The elongated main body portion generally defines a proximal extension arm that is angled with respect to a distal extent of the elongated main body portion. The elongated main body portion generally defines a substantially arcuate geometry. The substantially arcuate geometry extends over an arc of about 120° to about 180°. The elongated main body portion can generally flare out to define a junction region. The plurality of petals or fingers are detachably engaged to the elongated main body portion in the junction region. Further, the junction region generally includes a plurality of circumferentially spaced slots through which the plurality of petals or fingers are joined. The length of the plurality of petals or fingers can be adjustable by pulling the plurality of petals or fingers through the plurality of circumferentially spaced slots. In some exemplary embodiments, the plurality of petals or fingers can include features, e.g., notches, ridges, and the like, for releasably interlocking with the plurality of circumferentially spaced slots.

One or more intermediate structures can be positioned between the plurality of petals or fingers and the elongated main body portion. The plurality of petals or fingers generally define an inner region that is configured and dimensioned to accommodate the distal end of the surgical stapler. The plurality of petals or fingers can be detachably joined with respect to each other at the distal junction. The proximally-directed mounting member generally defines, e.g., a central channel for receipt of a distally-directed shaft member extending from the surgical stapler, a rod-like structure for receipt in a distally-directed channel associated with the surgical stapler, and the like. The distal junction and the proximally-directed mounting member associated with the plurality of petals or fingers can be adapted to disengage upon application of a requisite proximally-directed force to the elongated main body portion.

In accordance with embodiments of the present disclosure, exemplary methods for introducing a surgical stapler with respect to an anatomical region are provided that generally include providing a surgical stapler that includes a handle, a shaft, and a distally positioned stapling member. The exemplary methods generally include detachably mounting an introducer with respect to said surgical stapler. The exemplary introducer generally includes an elongated main body portion, one or more mounting mechanisms associated with the elongated main body portion, and a plurality of petals or fingers positioned at a distal end of the elongated main body portion. The one or more mounting mechanisms detachably mount with respect to the surgical stapler. The plurality of petals or fingers generally define a distal junction and a proximally-directed mounting member that detachably engage a distal portion of the surgical stapler. The exemplary methods generally further include adjusting a length of the plurality of petals or fingers to conform to a distal end of the surgical stapler.

The plurality of petals or fingers can define an inner region that can be configured and dimensioned to accommodate the distal end of the surgical stapler. The proximally-directed mounting member generally defines either a central channel for receipt of a distally-directed shaft member extending from the surgical stapler or a rod-like structure for receipt in a distally-directed channel associated with the surgical stapler. Adjusting the length of the plurality of petals or fingers generally includes pulling the plurality of petals or fingers through a plurality of circumferentially spaced slots located at a distal region of the elongated main body portion. The exemplary methods generally include disengaging the introducer from the surgical stapler. The introducer can be disengaged from the surgical stapler upon application of a proximally-directed force with respect to the elongated main body portion which causes the distal junction and the proximally-directed mounting member associated with the plurality of petals or fingers to disengage.

In accordance with embodiments of the present disclosure, a surgical stapler and an exemplary introducer assembly are provided in combination. The surgical stapler generally includes a handle, a shaft and a distally positioned stapling member. The exemplary introducer assembly generally includes an elongated main body portion, one or more mounting mechanisms associated with the elongated main body portion, and a plurality of petals or fingers positioned at a distal end of the elongated main body portion. The one or more mounting mechanisms can be detachably mounted with respect to the surgical stapler. The plurality of petals or fingers generally define a distal junction and a proximally-directed mounting member that are detachably engaged with respect to a distal portion of the surgical stapler. A length of the plurality of petals or fingers can be adjustable to conform to a distal end of the surgical stapler.

The plurality of petals or fingers associated with the introducer generally define an inner region that can be configured and dimensioned to accommodate the distal end of the surgical stapler. The plurality of petals or fingers can be detachably joined with respect to each other at the distal junction. The proximally-directed mounting member can define a central channel for receipt of a distally-directed shaft member extending from the surgical stapler or a rod-like structure for receipt in a distally-directed channel associated with the surgical stapler. The distal junction and the proximally-directed mounting member associated with the plurality of petals or fingers can be adapted to disengage upon application of a requisite proximally-directed force to the elongated main body portion.

In accordance with embodiments of the present disclosure, exemplary introducer assemblies for use with a surgical stapler are provided that generally include an elongated main body portion. The exemplary assemblies generally include one or more mounting mechanisms associated with the elongated main body portion. The one or more mounting mechanisms can be configured and dimensioned for detachably mounting with respect to the surgical stapler. The exemplary assemblies further generally include a plurality of petals or fingers defining a distal junction and a proximally-directed mounting member that can be configured and dimensioned to detachably engage a distal portion of the surgical stapler. The plurality of petals or fingers can be positioned at least in part radially outward of the proximally-directed mounting member so as to define an entry angle of the distal junction. The entry angle defined by the plurality of petals or fingers can be adjustable by adjusting a height of the proximally-directed mounting member. Increasing the height of the proximally-directed mounting member generally increases the entry angle. Decreasing the height of the proximally-directed mounting member generally decreases the entry angle.

In accordance with embodiments of the present disclosure, exemplary methods for introducing a surgical stapler with respect to an anatomical region are provided that generally include providing a surgical stapler that includes a handle, a shaft and a distally positioned stapling member. The exemplary methods generally include detachably mounting an introducer with respect to said surgical stapler. The exemplary introducer generally includes an elongated main body portion, one or more mounting mechanisms associated with the elongated main body portion, and a plurality of petals or fingers positioned at a distal end of the elongated main body portion.

The one or more mounting mechanisms generally detachably mount with respect to the surgical stapler. The plurality of petals or fingers generally define a distal junction and a proximally-directed mounting member that detachably engage a distal portion of the surgical stapler. The plurality of petals or fingers can be positioned at least in part radially outward of the proximally-directed mounting member so as to define an entry angle. The exemplary methods further generally include adjusting the entry angle defined by the plurality of petals or fingers by adjusting a height of the proximally-directed mounting member. Adjusting the entry angle generally further includes at least one of increasing the height of the proximally-directed mounting member to increase the entry angle and/or decreasing the height of the proximally-directed mounting member to decrease the entry angle.

In accordance with embodiments of the present disclosure, a surgical stapler and an exemplary introducer assembly are provided in combination. The surgical stapler generally includes a handle, a shaft and a distally positioned stapling member. The exemplary introducer assembly generally includes an elongated main body portion, one or more mounting mechanisms associated with the elongated main body portion, and a plurality of petals or fingers positioned at a distal end of the elongated main body portion. The one or more mounting mechanisms can be detachably mounted with respect to the surgical stapler. The plurality of petals or fingers generally define a distal junction and a proximally-directed mounting member that can be detachably engaged with respect to a distal portion of the surgical stapler. The plurality of petals or fingers can be positioned at least in part radially outward of the proximally-directed mounting member so as to define an entry angle. The entry angle defined by the plurality of petals or fingers can be adjustable by adjusting a height of the proximally-directed mounting member.

In accordance with embodiments of the present disclosure, exemplary introducer assemblies for use with a surgical stapler are provided that generally include an elongated main body portion. The exemplary assemblies generally include one or more mounting mechanisms associated with the elongated main body portion. The one or more mounting mechanisms can be configured and dimensioned for detachably mounting with respect to the surgical stapler. The exemplary assemblies further generally include a plurality of petals or fingers defining a distal junction and a proximally directed mounting member that is configured and dimensioned to detachably engage a distal portion of the surgical stapler. The proximally-directed mounting member can define means for detachably engaging a trocar, e.g., a notch located inside the proximally-directed mounting member.

In accordance with embodiments of the present disclosure, exemplary methods for introducing a surgical stapler with respect to an anatomical region are provided that generally include providing a surgical stapler. The surgical stapler generally includes a handle, a shaft and a distally positioned stapling member. The exemplary methods generally include detachably mounting an exemplary introducer with respect to said surgical stapler. The exemplary introducer generally includes an elongated main body portion, one or more mounting mechanisms associated with the elongated main body portion, and a plurality of petals or fingers positioned at a distal end of the elongated main body portion.

The one or more mounting mechanisms generally detachably mount with respect to the surgical stapler. The plurality of petals or fingers can define a distal junction and a proximally-directed mounting member that detachably engage a distal portion of the surgical stapler. The proximally-directed mounting member can define means for detachably engaging a trocar. The exemplary methods generally include detachably engaging a trocar with the proximally-directed mounting member. In addition, the exemplary methods generally include disengaging the trocar by disengaging the distal junction and the proximally-directed mounting member associated with the plurality of petals or fingers upon application of a requisite proximally-directed force to the elongated main body portion.

In accordance with embodiments of the present disclosure, a surgical stapler and an exemplary introducer assembly are provided in combination. The surgical stapler generally includes a handle, a shaft and a distally positioned stapling member. The exemplary introducer assembly generally includes an elongated main body portion, one or more mounting mechanisms associated with the elongated main body portion, and a plurality of petals or fingers positioned at a distal end of the elongated main body portion. The one or more mounting mechanisms can be detachably mounted with respect to the surgical stapler. The plurality of petals or fingers generally define a distal junction and a proximally-directed mounting member that can be detachably engaged with respect to a distal portion of the surgical stapler. The proximally-directed mounting member can define means for detachably engaging a trocar.

In accordance with embodiments of the present disclosure, exemplary introducer assemblies for use with a surgical stapler are provided that generally include an elongated main body portion. The exemplary assemblies generally include one or more mounting mechanisms associated with the elongated main body portion. The one or more mounting mechanisms can be configured and dimensioned for detachably mounting with respect to the surgical stapler. The exemplary assemblies generally include a plurality of petals or fingers defining a distal junction and a proximally-directed mounting member that can be configured and dimensioned to detachably engage a distal portion of the surgical stapler.

The exemplary assemblies further generally include means for insufflating air into a passage to an anatomical region. Means for insufflating air into the passage to the anatomical region can include, e.g., a hollow passage positioned within the elongated main body portion, a hollow passage positioned along an outer surface of the elongated main body portion. Means for insufflating air into the passage to the anatomical region further generally include a nipple configured and dimensioned to be connected to an insufflator bulb.

In accordance with embodiments of the present disclosure, exemplary methods for introducing a surgical stapler with respect to an anatomical region are provided that generally include providing a surgical stapler. The surgical stapler generally includes a handle, a shaft and a distally positioned stapling member. The exemplary methods generally include detachably mounting an introducer with respect to said surgical stapler. The exemplary introducer generally includes an elongated main body portion, one or more mounting mechanisms associated with the elongated main body portion, and a plurality of petals or fingers positioned at a distal end of the elongated main body portion. The exemplary introducer generally includes means for insufflating air into a passage to the anatomical region. The one or more mounting mechanisms detachably mount with respect to the surgical stapler. The plurality of petals or fingers generally define a distal junction and a proximally-directed mounting member that detachably engage a distal portion of the surgical stapler. The exemplary methods generally include insufflating air into the passage to the anatomical region.

In accordance with embodiments of the present disclosure, a surgical stapler and an introducer assembly are provided in combination. The surgical stapler generally includes a handle, a shaft and a distally positioned stapling member. The exemplary introducer assembly generally includes an elongated main body portion, one or more mounting mechanisms associated with the elongated main body portion, and a plurality of petals or fingers positioned at a distal end of the elongated main body portion. The exemplary introducer assembly generally includes means for insufflating air into a passage to an anatomical region. The one or more mounting mechanisms can be detachably mounted with respect to the surgical stapler. The plurality of petals or fingers generally define a distal junction and a proximally-directed mounting member that can be detachably engaged with respect to a distal portion of the surgical stapler.

Additional features, functions and benefits of the disclosed introducer device/assembly will be apparent from the figures which follow, particularly when viewed in conjunction with the accompanying description.

### BRIEF DESCRIPTION OF THE FIGURES

To assist those of ordinary skill in the art in making and using the introducer device and assembly of the present disclosure, reference is made to the accompanying figures, wherein:

FIG. 1 is a perspective view of an exemplary introducer assembly according to the present disclosure;

FIG. 2 is a further perspective view of an exemplary introducer assembly according to the present disclosure, rotated approximately 180° relative to the view of FIG. 1;

FIG. 3 is a perspective view of the exemplary introducer assembly of FIGS. 1 and 2 mounted with respect to an exemplary surgical stapler;

FIG. 4 is a further perspective view of an exemplary introducer assembly mounted with respect to an exemplary surgical stapler, rotated by approximately 90° relative to FIG. 3;

FIG. 5 is an enlarged view of the distal region of an exemplary introducer assembly according to the present disclosure mounted with respect to a surgical stapler;

FIGS. 6A and 6B are an enlarged and a cross-sectional view of a mounting member and a trocar of an exemplary introducer assembly; and

FIGS. 7A and 7B are side and top views of an exemplary introducer assembly and exemplary insufflation means mounted with respect to an exemplary surgical stapler.

### DESCRIPTION OF EXEMPLARY EMBODIMENT(S)

The disclosed devices, assemblies and methods advantageously facilitate introduction and/or positioning of a surgical stapler relative to a desired anatomical location. In addition, the disclosed devices, assemblies and methods permit a user to remotely detach the device/assembly from the surgical stapler, thereby permitting unobstructed operation of the surgical stapler in a desired manner. The disclosed devices, assemblies and methods also permit a user to extend the shaft of the surgical stapler, thereby permitting the surgical stapler to reach anatomical locations that otherwise may be hard to reach, while facilitating the introduction and/or positioning of the surgical stapler to these locations. The disclosed devices, assemblies and methods have wide ranging applicability and utility, including specifically applications throughout the gastrointestinal tract.

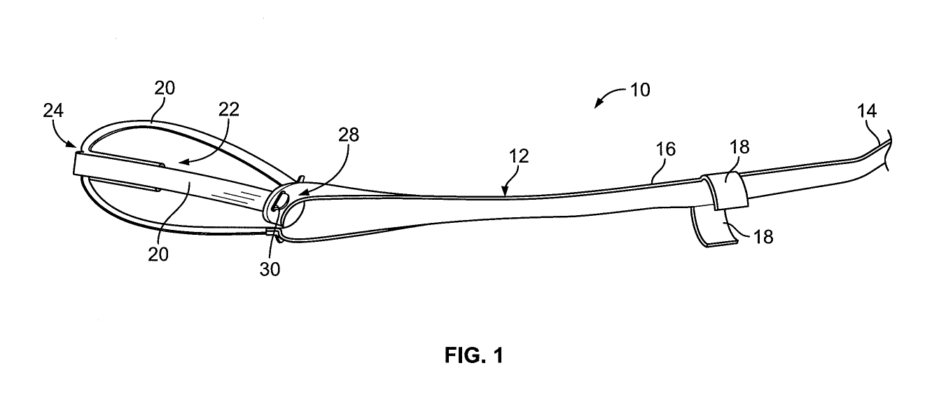

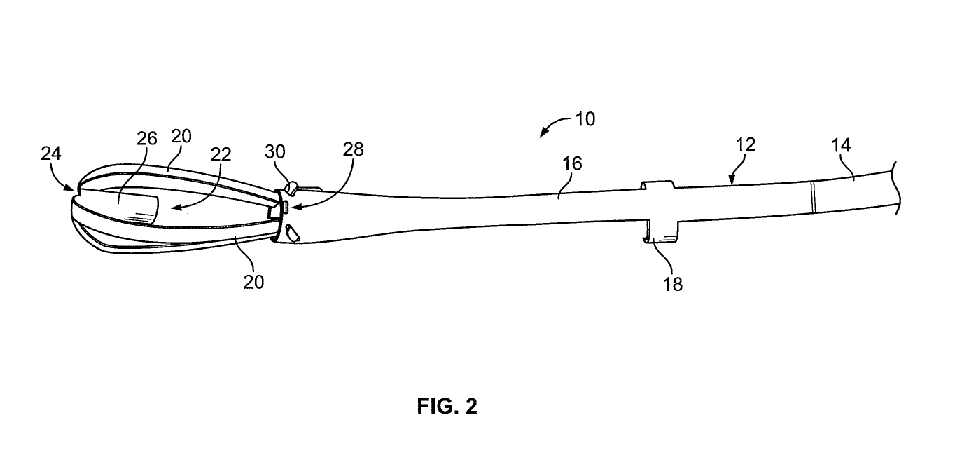

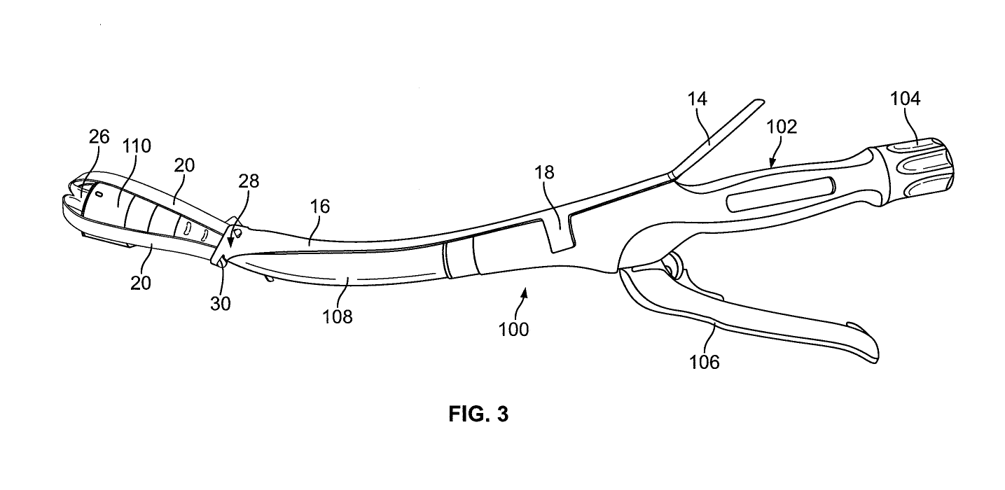

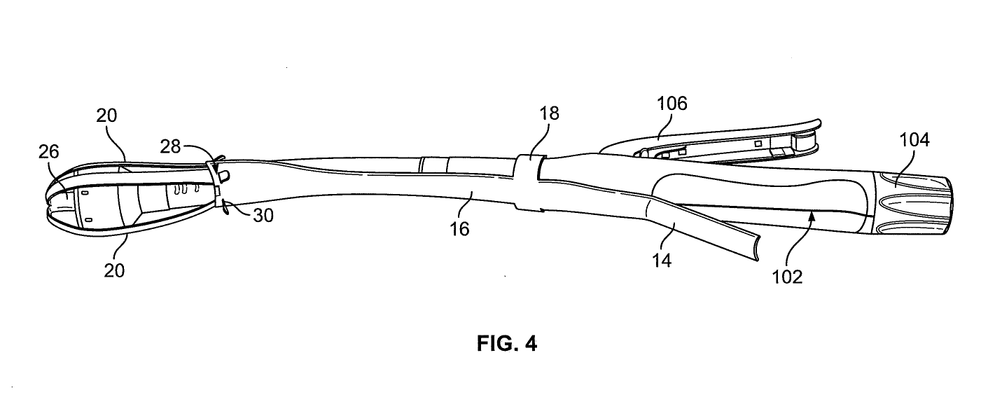

With initial reference to FIGS. 1 and 2, an exemplary introducer 10 is schematically depicted. Introducer 10 includes an elongated body member 12 that defines a proximal extension arm 14 that is angularly oriented relative to main body portion 16. The functionality of proximal extension arm 14 will be described in greater detail herein below with reference to FIGS. 3 and 4. The main body portion 16 defines a substantially arcuate geometry that, in use, is adapted to cooperate with a substantially cylindrical surgical stapler. In exemplary embodiments of the present disclosure, the main body portion 16 defines an arcuate body region that extends over an arc of about 120° to about 180°, although alternative arcuate dimensions may be employed. Indeed, the arcuate geometry of the disclosed main body portion 16 is generally selected so as to achieve two principal objectives: (i) cooperate/engage with the outer face of a cylindrical surgical stapler during stapler introduction/positioning, and (ii) permit remote detachment of the introducer from the surgical stapler, as desired by a user. Provided the foregoing principal objectives are satisfied, the arcuate geometry of the disclosed main body portion 16 may vary as to arcuate extent, radius of curvature and the like.

One or more mounting mechanisms are generally provided along the length of the main body portion 16 of the introducer 10 to facilitate detachable mounting of introducer 10 relative to a surgical stapler. Thus, in the exemplary embodiment of FIGS. 1 and 2, a pair of opposed, substantially C-shaped brackets 18 extend from main body portion 16 at an intermediate location at an intermediate location. The C-shaped brackets 18 generally exhibit sufficient flexibility/resilience to facilitate positioning around a substantially cylindrical shaft of a surgical stapler, yet sufficient strength/rigidity to ensure that introducer 10 is securely retained on or with respect to the surgical stapler until detachment is desired. In preferred embodiments of the present disclosure, the mounting mechanism(s), e.g., C-shaped brackets 18, are integrally formed with main body portion 16, e.g., as a single molded unit. However, the present disclosure also encompasses designs/implementations wherein the mounting mechanism(s) are separately formed/fabricated and joined to the main body portion 16, e.g., through sonic welding, adhesive, pin/slot, bayonet lock or other joining technology. In addition, although only a single pair of brackets 18 are depicted with respect to the exemplary introducer 10 of FIGS. 1 and 2, it should be understood that the present disclosure is not limited to such implementation, and that additional mounting brackets (or other mounting mechanisms) may be positioned along the length extent of the main body portion 16 without departing from the spirit or scope of the present disclosure.

With further reference to FIGS. 1 and 2, exemplary introducer 10 includes a plurality of petals or fingers 20 that extend from the distal end of main body portion 16. In the exemplary embodiment of FIGS. 1 and 2, introducer 10 includes four (4) petals/fingers 20, but the present disclosure is not limited by or to such exemplary implementation. Thus, as used herein, the term “plurality” of petals/fingers encompasses any design that includes two or more petals/fingers that deliver the functionalities described herein. In particular, petals/fingers 20 define an inner region 22 that is configured/dimensioned to accommodate the distal end of a surgical stapler, including specifically the staple cartridge of a surgical stapler. In addition, petals/fingers 20 are substantially joined at a distal junction 24 and, from such distal junction 24, define a proximally-directed mounting member 26.

In an alternative embodiment, the disclosed introducer 10 may include or cooperate with one or more intermediate structures, e.g., extension member(s) (not pictured). For example, one or more intermediate structures may be positioned between the disclosed petals/fingers 20 and the main body portion 16, e.g., such that the petals/fingers 20 are mounted with respect to the intermediate structure(s). Of note, the intermediate structure(s) may include structural features and/or functionalities that facilitate interaction with a surgical stapler, e.g., mounting mechanism(s) for detachably securing the disclosed introducer with respect to a surgical stapler.

In exemplary embodiments of the present disclosure, mounting member 26 defines a central channel that is configured and dimensioned to receive a distally-directed shaft member extending from the surgical stapler. Alternatively, mounting member 26 may define a rod-like structure that is configured and dimensioned for receipt in a distally-directed channel associated with the surgical stapler. In any case, mounting member 26 is adapted to detachably secure the distal end of introducer 10 with respect to the distal end of a surgical stapler. Various techniques may be employed to combine the plurality of petals/fingers 20 at distal junction 24 and to define mounting member 26 therefrom. For example, the petals/fingers 20 may be adhered with respect to each other using conventional adhesives. Alternatively, one or more banding members may be positioned around the petals/fingers 20 in the vicinity of distal junction 24 and/or the proximally-directed mounting member 26. Additionally, the plurality of petals may be mounted with respect to the main body portion by way of intermediate structure(s), e.g., for purposes of reaching anatomical locations that may otherwise be difficult to reach.

Petals/fingers 20 are also joined to main body portion 16 at proximal junction region 28. In the exemplary embodiment of FIGS. 1 and 2, proximal junction region 28 includes circumferentially spaced mounting slots 30 formed toward the distal end of main body portion 16. Indeed, as shown in FIGS. 1 and 2, main body portion 16 flares to a greater arcuate extent at its distal end, thereby accommodating four (4) circumferentially spaced mounting slots for petals/fingers 20. Thus, according to the exemplary implementation of FIGS. 1 and 2, petals/fingers 20 are fed through the mounting slots 30 and anchored therewithin. Additional steps may be taken to ensure that the petals/fingers 20 do not disengage from main body portion 16, e.g., application of an adhesive or the like. Of note, the petals/fingers 20 are generally fabricated from a material that exhibits greater flexibility than main body portion 16. Although plastics are preferred, it is contemplated that appropriate metals may be employed in fabricating introducer 10, as will be apparent to persons of skill in the art.

Thus, the disclosed introducer 10 exhibits “tear-away” functionality in that, once the mounting member 26 is mounted with respect to the distal end of a surgical stapler, a proximally-directed force applied to main body portion 16, e.g., by grasping proximal extension arm 14, will overcome the banding of the petals/fingers 20 at distal junction and in the region of mounting member 26. In this way, the plurality of petals/fingers 20 become disengaged from each other and from the distal end of the surgical stapler. Once disengaged from each other and from the surgical stapler, the petals/fingers 20 are free to slide proximally along the exterior of the surgical stapler. In addition, application of a proximally-directed force to the main body portion 16, e.g., to proximal extension arm 14, is generally effective to disengage the mounting mechanism(s) associated with the main body portion 16, e.g., C-shaped brackets 18, from the surgical stapler.

Turning to FIGS. 3-5, the exemplary introducer 10 of FIGS. 1 and 2 is detachably mounted with respect to a conventional surgical stapler according to an illustrative implementation of the present disclosure. Thus, as shown in FIGS. 3 and 4, conventional surgical stapler 100 includes a handle 102 that includes an approximating knob 104 and an actuating trigger 106. A curved shaft 108 extends from handle 102 and cooperates with a staple cartridge 110 positioned at a distal end thereof. Although surgical stapler 100 is illustrative of the types of surgical staplers with which the disclosed introducer device/assembly/method may be employed, it is neither exclusive nor limiting of the potential applications and/or uses of the disclosed introducer.

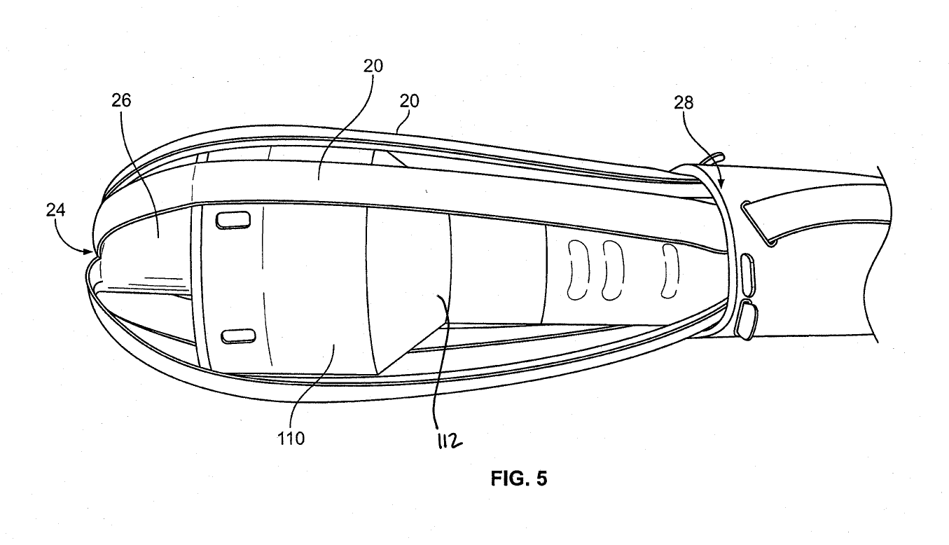

As shown in FIGS. 3 and 4, introducer 10 is detachably mounted with respect to stapler 100 in three ways: (i) the arcuate geometry of main body portion 16 substantially conforms to the geometry of shaft 108; (ii) C-shaped brackets 18 engage stapler 100 in a substantially cylindrical region of handle 102; and (iii) mounting member 26 detachably engages a distally-facing cooperating structure associated with staple cartridge 110 (see FIG. 5). Thus, in use the disclosed introducer 10 is generally detachably mounted with respect to a surgical stapler, e.g., surgical stapler 100. The petals/fingers 20 surround the distal end of surgical stapler 100, particularly the substantially flat face of staple cartridge 110, thereby providing a substantially flexible and tapered guide geometry (when viewed from the distal end of the stapler/introducer assembly) that assists in introducing and navigating surgical stapler 100 to a desired anatomical location. In particular, in advancing the surgical stapler 100 to a desired stapling site, the petals/fingers 20 of the introducer 10 help to guide the stapler 100 past potential obstructions and/or anatomical irregularities.

Once surgical stapler 100 is brought to a desired anatomical location, the user can easily disengage introducer 10 from surgical stapler 100 and withdraw the introducer from the surgical field. In particular, the user disengages introducer 10 from surgical stapler 100 by pulling proximally on main body portion 16 of introducer 10, e.g., by grasping proximal extension arm 14, thereby disengaging mounting member 26 from the stapler cartridge 110, disengaging C-shaped brackets 18 from handle 102, and disengaging the arcuate main body portion 16 overall from the surgical stapler shaft 108. Thus, in toto, the disclosed petals/fingers 20 function to “tear away” from the staple cartridge 110 so as to permit withdrawal from the surgical field. Of note, the angled geometry of proximal extension arm 14 facilitates ready access to and grasping thereof by a user when disengagement of introducer 10 from stapler 100 is desired.

Still with reference to FIG. 5, rather than implementing varying introducers 10 based on a particular stapler head and/or cartridge size, e.g., 24 cm, 29 cm, 32 cm, and the like, the exemplary introducer 10 can be a substantially one-size-fits-all model. In particular, as described above, the plurality of petals or fingers 20 detachably engage the proximal junction region 28 of the elongated main body portion 16 by being fed through and anchored within mounting slots 30. The inner surface of the plurality of petals or fingers 20, i.e., the surface facing the surgical stapler 100, can define features, e.g., notches, ridges, and the like, for releasably interlocking the plurality of petals or fingers 20 within the plurality of mounting slots 30.

As would be understood by those of ordinary skill in the art, pulling the plurality of petals or fingers 20 through the mounting slots 30 releasably interlocks the plurality of petals or fingers 20, while shortening the length of the plurality of petals or fingers 20 positioned around the staple cartridge 110 and the stapler neck 112. The plurality of petals or fingers 20 can thereby be tightened such that the proximal junction region 28 is pulled up against and abuts the stapler neck 112. By tightening or loosening the plurality of petals or fingers 20, the introducer 10 can be adapted to conform to a variety of stapler head and/or cartridge sizes. In addition, the configuration and/or size of the mounting member 26 may be modified as needed to detachably engage the distally-facing cooperating structure associated with various staple cartridge 110 models.

The adjustable length of the plurality of petals or fingers 20 can further be implemented to adjust the entry angle of the introducer 10, i.e., the degree of taper of the plurality of petals or fingers 20. In particular, the plurality of petals or fingers 20 are generally positioned at least in part radially outward of the distal junction 24, thereby defining an entry angle prior to detachably engaging the proximal junction region 28 of the elongated main body portion 16. By adjusting the length of the plurality of petals or fingers 20, the height of the mounting member 26 can be adjusted. For example, as illustrated in FIG. 5, the mounting member 26 can initially be fabricated to define a predetermined height. Thus, the mounting member 26 extends from the staple cartridge 110 and the plurality of petals or fingers 20 extending radially outward of the distal junction 24 engage the proximal junction region 28 to define a substantially acute entry angle and/or angle taper.

As described above, the plurality of petals or fingers 20 join at distal junction 24 to define the mounting member 26. For example, if the introducer 20 includes four petals or fingers 20, each of the four petals or fingers 20 defines a fourth of the circumference of the mounting member 26. The plurality of petals or fingers 20 joined to define the mounting member 26 can be joined by, e.g., perforated lines, seams, and the like, such that the mounting member 26 can be “torn” apart upon a requisite proximally-directed force applied to the plurality of petals or fingers 20. Thus, although initially defining an acute entry angle, by applying a requisite proximally-directed force to the plurality of petals or fingers 20 and pulling the plurality of petals or fingers 20 through the mounting slots 30, the height of the mounting member 26 can be gradually decreased as the plurality of petals or fingers 20 “tear apart”. The decreased height of the mounting member 26 adjusts the extent to which the plurality of petals or fingers 20 extend radially from the distal junction 24, thereby defining a more blunt entry angle. The adjustable entry angle generally aids in passing the introducer 10 through lumens having both wide and narrow passages.

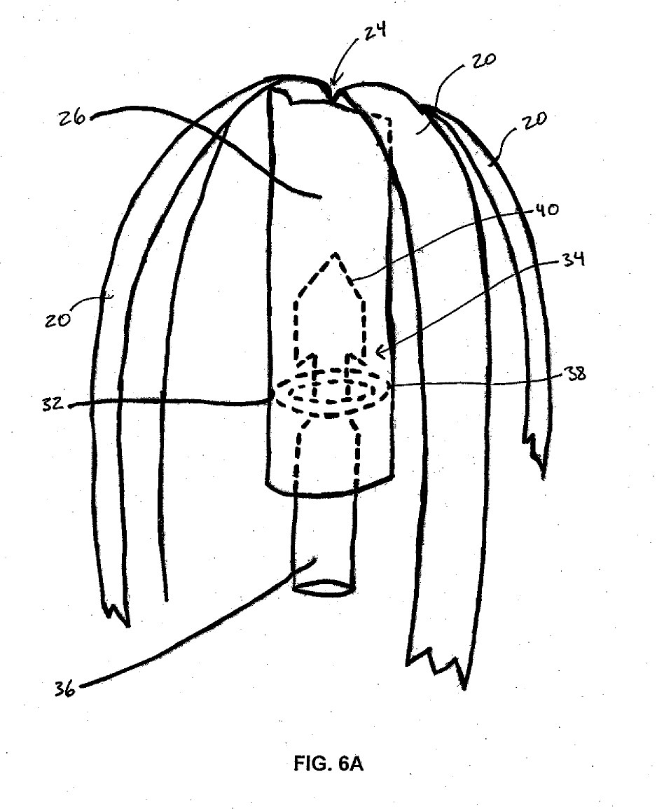

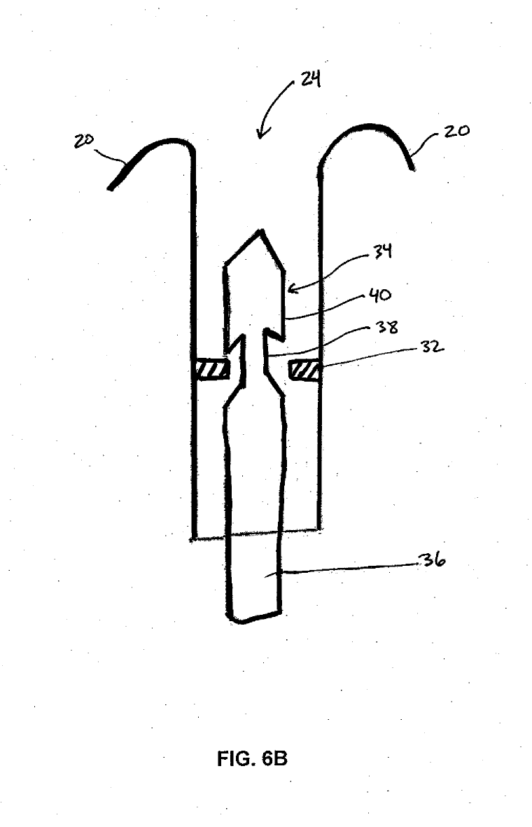

Turning now to FIGS. 6A and 6B, an exemplary mounting member 26 is shown, including a central channel and means for detachably engaging an attachable trocar 34. The trocar 34 generally includes a body portion 36, a head portion 40, and a connection portion 38 connecting the body portion 36 and the head portion 40. The trocar 34 can be attached to a distally-directed shaft member and/or trocar extending from the surgical stapler 100 (not shown). The trocar 34 can further be positioned within the central channel of the mounting member 26. A breakable connection 32, e.g., a notch, a lip, an edge, petals, and the like, can be positioned between the trocar 34 and the mounting member 26. In particular, the trocar 34 and the breakable connection 32 can be configured and dimensioned such that the body portion 36 and the head portion 40 of the trocar 34 generally cannot pass through the inner perimeter of the breakable connection 32. As would be understood by those of ordinary skill in the art, the trocar 34 can thereby be maintained within the central channel of the mounting member 36 and prevented from undesired motion. In addition, the breakable connection 32 can be positioned within the mounting member 26 such that the trocar 34 does not protrude beyond the distal junction 24. In some exemplary embodiments, the trocar 34 may include, e.g., a notch, and the like, configured and dimensioned to receive the breakable connection 32.

As an example, the distally-directed shaft member can be extended and the attachable trocar 34 can be attached to the breakable connection 32 within the mounting member 26. The distally-directed shaft member may remain partially open, thereby anchoring the mounting member 26 to the surgical stapler 100 within the stapler head. In addition, the plurality of petals or fingers 20 may be tightened, as described above, to wedge the proximate junction region 28 of the elongated main body portion 16 against the stapler neck 112 to ensure that the surgical stapler 100 and the introducer 10 are securely interlocked.

Once the surgical stapler 100 has been securely positioned, the distally-directed shaft member can be fully closed to break the breakable connection 32 between the trocar 34 and the mounting member 26. The elongated main body portion 16 can further be proximally pulled, thereby “tearing away” the plurality of petals or fingers 20 and disengaging the mounting member 26 from the stapler cartridge 110. In some exemplary embodiments, rather than maintaining the distally-directed shaft member in a partially open position, the distally-directed shaft member can be fully closed and the breakable connection 32 can be “torn away” simultaneously to the “tearing away” of the plurality of petals or fingers 20. The distally-directed shaft member with the attachable trocar 34 can be extended to create the desired opening in the bowel. The trocar 34 can then be removed such that the anvil can be attached to the distally-directed shaft member.

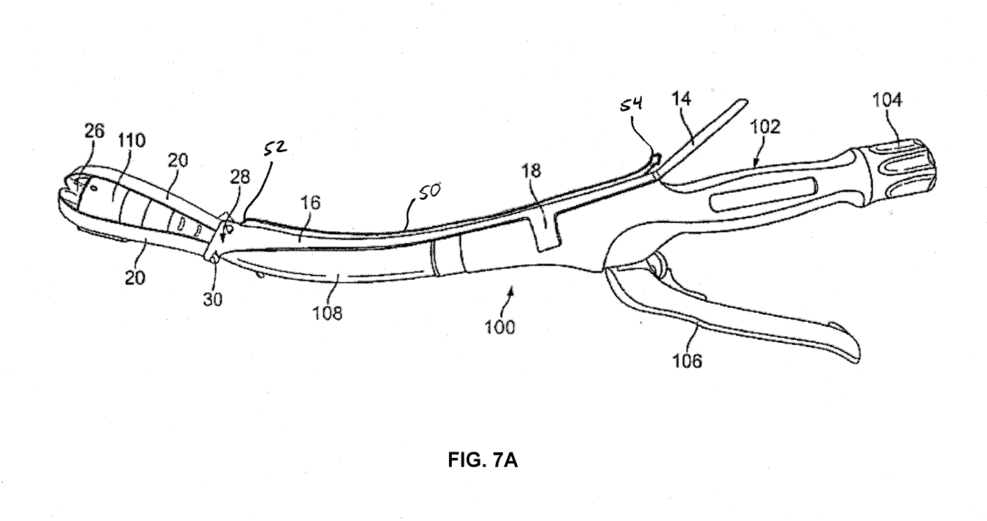

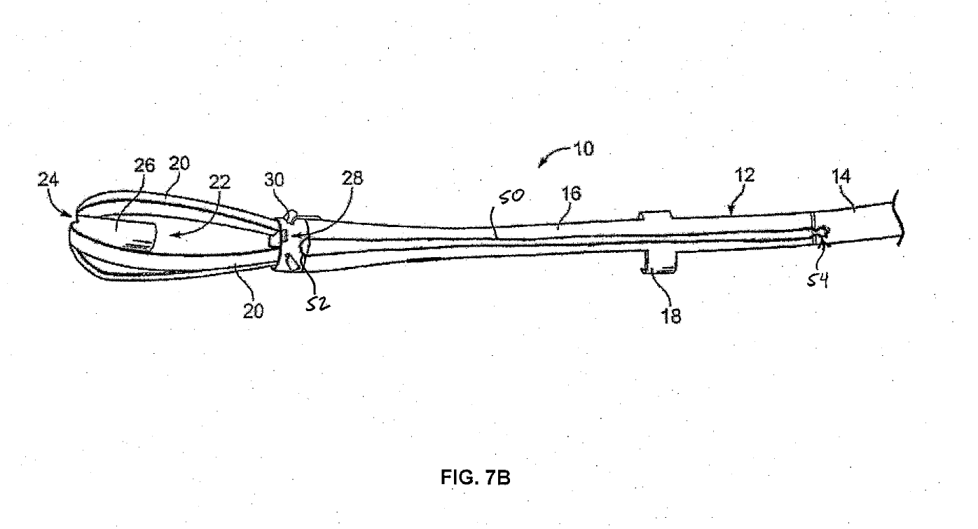

Turning now to FIGS. 7A and 7B, an exemplary introducer 10 is provided that includes means for insufflating air into a lumen, e.g., a passage to an anatomical region. Means for insufflating air into a lumen generally include a hollow channel 50, e.g., a passage, and the like, including an opening 52 at a distal end and a nipple 54 at a proximal end. The hollow channel 50 can be configured and dimensioned to extend from the proximal extension arm 14 to the proximal junction region 28. Further, the hollow channel 50 can be positioned, e.g., within the elongated main body portion 16, along an outer surface of the elongated main body portion 16, and the like. The nipple 54 can be configured and dimensioned to be connected to an insufflator bulb, e.g., an insufflator bulb implemented on blood pressure cuffs, for insufflating the air. The hollow passage 50 can be utilized to insufflate air into the bowel similar to, for example, the methods implemented to insufflate air during an endoscopy. As would be understood by those of ordinary skill in the art, means for insufflating air into the lumen can distend the bowel and/or smooth out the folds of the lumen, thereby simplifying the introduction and passage of the introducer 10 and/or the surgical stapler 100.

The disclosed introducer offers significant advantages for introducing and positioning surgical staplers in a desired anatomical location. In addition, the disclosed introducer is designed such that modifications to conventional surgical staplers are unnecessary to gain the clinical benefits associated therewith. Indeed, the disclosed introducer is susceptible to widespread adoption and use, thereby overcoming a fundamental issue encountered in many surgical applications, namely reliable introduction and positioning of the surgical stapler despite potential irregularities and/or anomalies in a patient's anatomy.

Although the present disclosure has been described with reference to exemplary embodiments and implementations thereof, it is to be understood that the present disclosure is not limited by or to such exemplary embodiments and/or implementations. Rather, the present disclosure is susceptible to various modifications, variations and/or enhancements without departing from the spirit and/or scope of the present disclosure. Thus, the present disclosure expressly encompasses such potential modifications, variations and/or enhancements, as will become apparent to persons of skill in the art from the present disclosure.

1. An introducer assembly for use with a surgical stapler, comprising:
a. an elongated main body portion;
b. one or more mounting mechanisms associated with the elongated main body portion, said one or more mounting mechanisms being configured and dimensioned for detachably mounting with respect to the surgical stapler; and
c. a plurality of petals or fingers defining a distal junction and a proximally-directed mounting member that is configured and dimensioned to detachably engage a distal portion of the surgical stapler,
wherein a length of the plurality of petals or fingers is adjustable to conform to a distal end of the surgical stapler.

2. The introducer assembly of claim 1, wherein the elongated main body portion defines a proximal extension arm that is angled with respect to a distal extent of the elongated main body portion.

3. The introducer assembly of claim 1, wherein the elongated main body portion defines a substantially arcuate geometry.

4. The introducer assembly of claim 3, wherein the substantially arcuate geometry extends over an arc of about 120° to about 180°.

5. The introducer assembly of claim 1, wherein the elongated main body portion flares out to define a junction region, and wherein the plurality of petals or fingers are detachably engaged to the elongated main body portion in the junction region.

6. The introducer assembly of claim 5, wherein the junction region includes a plurality of circumferentially spaced slots through which the plurality of petals or fingers are joined, and wherein the length of the plurality of petals or fingers is adjustable by pulling the plurality of petals or fingers through the plurality of circumferentially spaced slots.

7. The introducer assembly of claim 6, wherein the plurality of petals or fingers include features for releasably interlocking with the plurality of circumferentially spaced slots.

8. The introducer assembly of claim 1, wherein the proximally-directed mounting member defines means for detachably engaging a trocar.

9. The introducer assembly of claim 1, wherein one or more intermediate structures are positioned between the plurality of petals or fingers and the elongated main body portion.

10. The introducer assembly of claim 1, wherein the plurality of petals or fingers define an inner region that is configured and dimensioned to accommodate a distal end of the surgical stapler.

11. The introducer assembly of claim 1, wherein the plurality of petals or fingers are detachably joined with respect to each other at the distal junction.

12. The introducer assembly of claim 1, wherein the proximally-directed mounting member defines either (i) a central channel for receipt of a distally-directed shaft member extending from the surgical stapler, or (ii) a rod-like structure for receipt in a distally-directed channel associated with the surgical stapler.

13. The introducer assembly of claim 1, wherein the plurality of petals or fingers are positioned at least in part radially outward of the proximally-directed mounting member so as to define an entry angle; and wherein the entry angle defined by the plurality of petals or fingers is adjustable by adjusting a height of the proximally-directed mounting member.

14. A method for introducing a surgical stapler with respect to an anatomical region, comprising:
a. providing a surgical stapler that includes a handle, a shaft and a distally positioned stapling member;
b. detachably mounting an introducer with respect to said surgical stapler, said introducer including (i) an elongated main body portion, (ii) one or more mounting mechanisms associated with the elongated main body portion; and (iii) a plurality of petals or fingers positioned at a distal end of the elongated main body portion;
wherein said one or more mounting mechanisms detachably mount with respect to the surgical stapler, and wherein said plurality of petals or fingers define a distal junction and a proximally-directed mounting member that detachably engage a distal portion of the surgical stapler; and
c. adjusting a length of the plurality of petals or fingers to conform to a distal end of the surgical stapler.

15. The method of claim 14, wherein the plurality of petals or fingers define an inner region that is configured and dimensioned to accommodate a distal end of the surgical stapler.

16. The method of claim 14, wherein the proximally-directed mounting member defines either (i) a central channel for receipt of a distally-directed shaft member extending from the surgical stapler, or (ii) a rod-like structure for receipt in a distally-directed channel associated with the surgical stapler.

17. The method of claim 14, wherein adjusting the length of the plurality of petals or fingers further comprises pulling the plurality of petals or fingers through a plurality of circumferentially spaced slots located at a distal region of the elongated main body portion.

18. The method of claim 14, further comprising disengaging the introducer from the surgical stapler.

19. The method of claim 18, wherein the introducer is disengaged from the surgical stapler upon application of a proximally-directed force with respect to the elongated main body portion which causes the distal junction and the proximally-directed mounting member associated with the plurality of petals or fingers to disengage.

20. In combination:
a. a surgical stapler that includes a handle, a shaft and a distally positioned stapling member;
b. an introducer assembly including (i) an elongated main body portion, (ii) one or more mounting mechanisms associated with the elongated main body portion; and (iii) a plurality of petals or fingers positioned at a distal end of the elongated main body portion;
wherein said one or more mounting mechanisms are detachably mounted with respect to the surgical stapler,
wherein said plurality of petals or fingers define a distal junction and a proximally-directed mounting member that are detachably engaged with respect to a distal portion of the surgical stapler, and
wherein a length of the plurality of petals or fingers is adjustable to conform to a distal end of the surgical stapler.
wherein the proximally-directed mounting member defines means for detachably engaging a trocar.
